(19) United States
(12) Patent Application Publication
(10) Pub. No.: US 2021/0070148 A1
(43) Pub. Date: Mar. 11, 2021

(54) IN-CAR ENVIRONMENT PURIFICATION DIFFUSER

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 17/102,236

(22) Filed: Nov. 23, 2020

Related U.S. Application Data
(63) Continuation-in-part of application No. 16/259,833, filed on Jan. 28, 2019.
Application No. 16/259,833 is a continuation-in-part of application No. 16/157,994, filed on Oct. 11, 2018.

(51) Int. Cl. B60H 3/00; B05B 7/24; A61L 9/14

(52) U.S. Cl. CPC B60H 3/0007; B05B 7/2491; A61L 2209/134; A61L 9/14; A61L 2209/11; B60H 3/0035

(57) ABSTRACT

An in-car environment purification diffuser includes a spray cover combined with a tank cover and covered on a slot of a water tank. The oscillation space in the water tank is formed with a tubular protrusion of an air flow channel. When the diffuser is dumped, the liquid in the water tank is less likely to seep into the diffuser or the car. The oscillator mounted in the water tank is coated with an acid-and-alkaline-resistant coating. The user can add safe cleaning liquid such as alkaline hydroxide ionized water into the oscillation space and atomize it as deodorization, sterilization, disinfection, etc. for the car's environmental purification. A light-permeable spray cover with an umbrella-shaped panel is to shield the tank cover. The spray cover is like a paper umbrella with a decorative effect. The umbrella-shaped panel with spray effect is decorative and can create a better atmosphere in the car.

This application is a Continuation-in-Part of application Ser. No. 16/259,833 filed Jan. 28, 2019.

### BACKGROUND OF THE INVENTION

### 1. Field of the Invention

The present invention relates to diffuser technology and more particularly, to an in-car environment purification diffuser, which converts liquid to fine vapor by oscillation.

### 2. Description of the Related Art

Existing diffuser devices are used for washing, deodorizing, sterilizing, disinfecting and other functions. These diffuser devices use electronic parts, electrical components, wires, etc. When the diffuser device filled with chemical liquid is dumped, the chemical liquid easily penetrates into the diffuser device and damages or affects the operation performance of these electronic parts or electrical components. The chemical liquid overflowing the diffuser device is also easy to cause environmental pollution and harm the human body, and it is not safe to use. Moreover, the acid-alkaline liquid in the water tank of the existing diffuser device is easy to corrode the oscillating sheet, causing the positive and negative electrode layers to conduct with each other and damage, and the problem of forming a fine vapor cannot produce a better environmental effect.

### SUMMARY OF THE INVENTION

The present invention has been accomplished under the circumstances in view. It is one object of the present invention to provide an in-car environment purification diffuser, which is so configured that when the in-car environment purification diffuser is dumped, the liquid in the diffuser is less likely to flow into the interior and the car environment, which helps to avoid damage to the circuit components in the diffuser and maintain the clean and safe environment in the car.

The present invention provides an in-car environment purification diffuser that can use a highly alkaline ionized water liquid, and the generated alkaline ionized water spray cleans, sterilizes, deodorizes, and disinfects the environment in the car, so that the environment in the car is safe and does not harm human health.

The present invention provides an in-car environment purification diffuser that includes decoration or lighting combined with spray effects to create a better atmosphere in the car.

In a preferred embodiment of the present invention, the in-car environment purification diffuser comprises a bottom housing, an electric fan, a water tank, an oscillation device, a spray cover, and a control unit. The bottom housing comprises an accommodation chamber defined inside, a first air inlet formed on the bottom side of the accommodation chamber, an opening formed in an upper edge of the accommodation chamber. The water tank comprises a slot, an oscillation space, a tank bottom, a perforation, a tank cover, and an air flow channel. The slot is formed on the upper side of the water tank. The oscillation space is defined in the water tank for holding a liquid. The tank bottom is provided with a perforation in communication with the oscillation space. The air flow channel has one side thereof protruding into the oscillation space of the water tank to form an inner vent, and an opposite side thereof extending out of the tank bottom to form an outer vent. The electric fan is mounted in the outer vent. In some embodiments, the diameter of the outer vent is larger than that of the inner vent, so that the airflow produced by the electric fan can flow from the outer vent through the inner vent into the oscillation space at a relatively higher speed and higher pressure.

The water tank is mounted in the accommodation chamber. The outer vent of the air flow channel and the electric fan are disposed in communication with the first air inlet in the accommodation chamber. The first air inlet provides a path for the electric fan to inhale external air.

The tank cover comprises a shield and a first exhaust hole. The shape of the tank cover matches the structure of the inner vent in the water tank. The shield forms a concave shape on an inner side of the peripheral edge of the tank cover. The inner side of the shield concave shape forms a sheltered space. The first exhaust hole is formed on an opposite side of the tank cover. The tank cover is detachably covered on the slot. The shield of the tank cover is covered over the inner vent. The inner vent and the first exhaust hole are commonly disposed in communication with the oscillation space.

The oscillation device is mounted in the perforation and used to vibrate and atomize the liquid in the oscillation space.

The spray cover comprises a spray hole. The spray cover is detachably mounted on the opening of the bottom housing to shield the tank cover, so that the spray hole communicates with the first exhaust hole and the oscillation space to form a spray outlet path.

The control unit is mounted in the accommodation chamber at one side of the tank bottom. The control unit comprises a circuit device such as a printed circuit board assembly, and a circuit for driving the oscillation device. The control unit is electrically connected with the oscillation device, the electric fan, or other lamps, electronics or appliances used in the in-car environment purification diffuser. It is used to control the switching or various action functions of the oscillation device or the electric fan or other lamps, electronics or appliances.

In this way, connect a power supply to the control unit, command to turn on the oscillation device to oscillate the liquid in the oscillation space (such as alkaline ionized water or various environmental cleaning and purification agents, etc., or add various natural sterilization and deodorant agents to the liquid for application) into atomized mist (fine water molecules). At the same time, the electric fan continuously sends the air flow from the first air inlet to the outer vent to passthrough the inner vent of the air flow channel. The airflow is blocked by the shield from backflow, and it flows in the oscillation space without directly flowing out of the first exhaust hole. It can effectively transport atomized alkaline ionized water (in the embodiment, the pH value of the hydroxide ionized water electrolyzed by pure water is about 10 to 12.5) and various mists (fine water molecules) from the oscillation space through the first exhaust hole and the spray cover to the car internal space or the outside environment for use in sterilization, cleaning, deodorization, virus elimination, or air purification. The light-permeable spray cover is light permeable and provided with an umbrella-shaped panel to shield the tank cover. The spray cover is shaped like a paper umbrella with a decorative effect. The umbrella-shaped panel with spray effect is very decorative and can create a better atmosphere in the car.

The present invention provides an embodiment. When the in-car environment purification diffuser is dumped, the liquid in the diffuser is less likely to flow into the interior and the car environment, which helps to avoid damage to the circuit components in the diffuser and maintain the clean and safe environment in the car.

The present invention provides an embodiment, wherein the oscillator of the oscillation device is coated with an acid-and-alkaline-resistant coating, so that an alkaline ionized water liquid can be used in the diffuser, and the generated alkaline ionized water spray can clean, sterilize, deodorize, and disinfect the environment in the car, which is safe and does not harm human health.

The present invention provides an embodiment, wherein the spray cover is light permeable and provided with an umbrella-shaped panel decoration that resembles a Hakka paper umbrella with a sense of beauty and charm, with lighting and spray effects to create a better atmosphere in the car.

Other advantages and features of the present invention will be fully understood by reference to the following specification in junction with the accompanying drawings, in which like reference signs denote like components of structure.

### BRIEF DESCRIPTION OF THE DRAWINGS

FIG. 1 is an elevational view of an in-car environment purification diffuser in accordance with the present invention.

FIG. 2 is an exploded view of the in-car environment purification diffuser in accordance with the present invention.

FIG. 3 is another exploded view of the in-car environment purification diffuser in accordance with the present invention.

FIG. 4 is a sectional side view of the in-car environment purification diffuser shown in FIG. 1.

FIG. 5 is a sectional elevational view of the in-car environment purification diffuser shown in FIG. 1.

FIG. 6 is a sectional view of the tank cover.

FIG. 7 is a sectional view of the spray cover.

### DETAILED DESCRIPTION OF THE PREFERRED EMBODIMENT

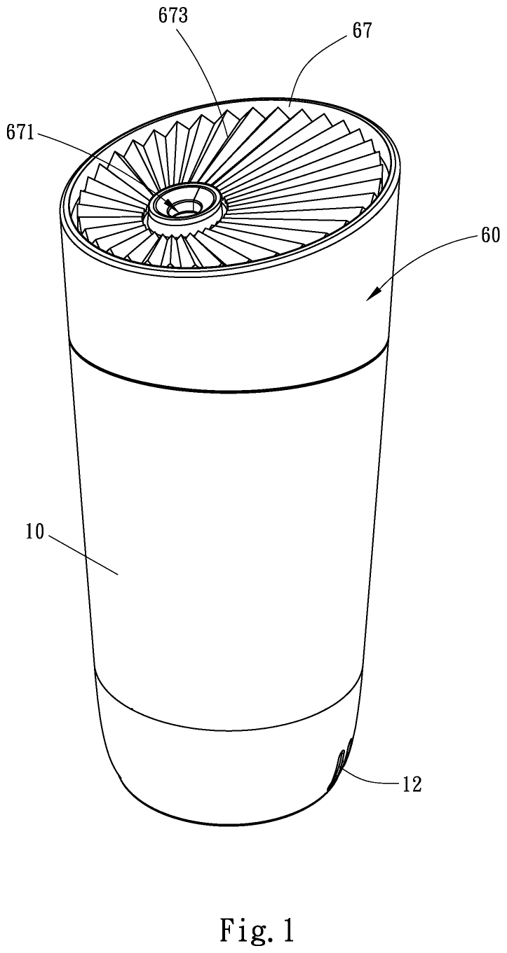

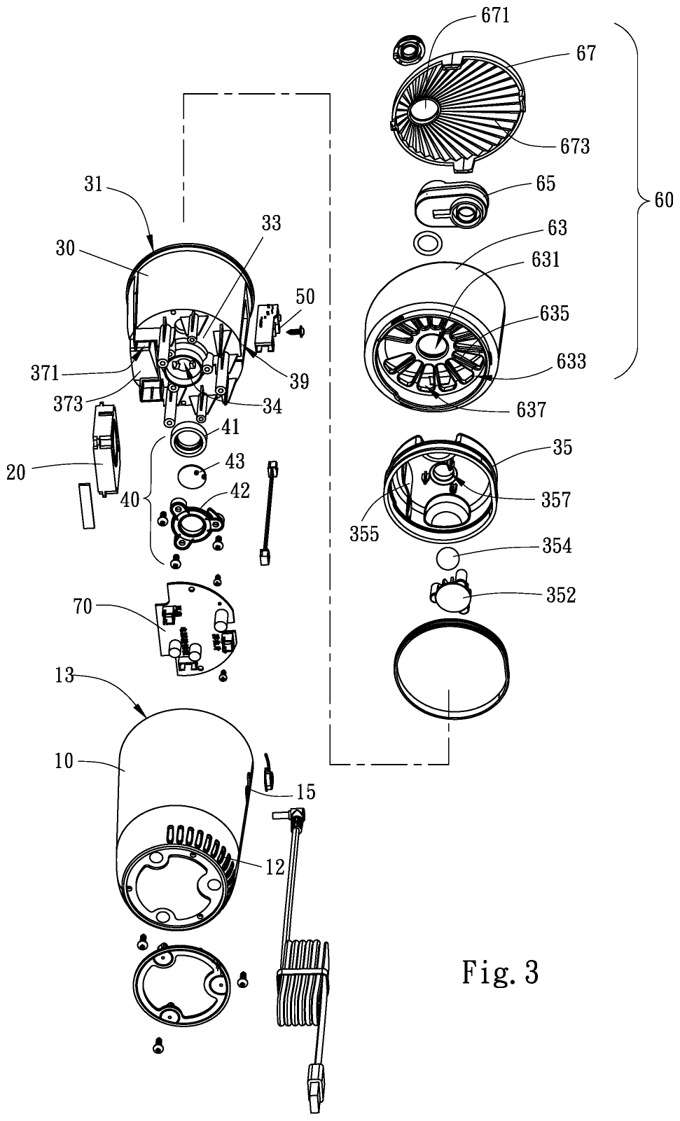

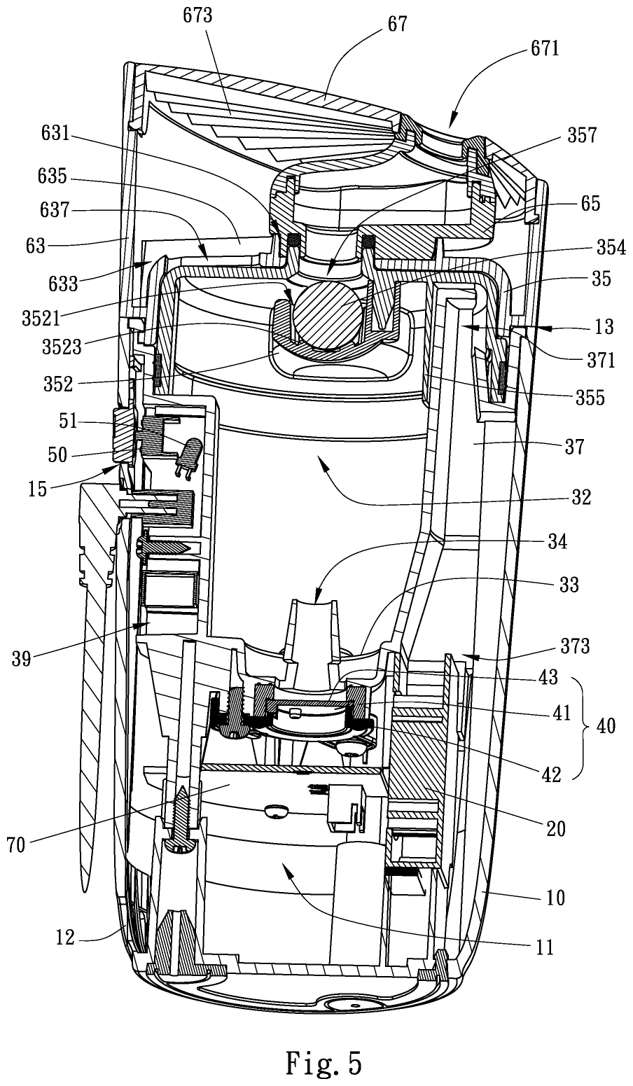

Referring to FIGS. 1-5, an in-car environment purification diffuser in accordance with the present invention comprises a bottom housing 10, an electric fan 20, a water tank 30, an oscillation device 40, a spray cover 60 and a control unit 70.

The bottom housing 10 comprises an accommodation chamber 11 defined inside, a first air inlet 12 provided on the bottom side of the accommodation chamber 11, and an opening 13 formed on the upper edge of the accommodation chamber 11.

The water tank 30 comprises a slot 31, an oscillation space 32, a tank bottom 33, a perforation 34, a tank cover 35, and an air flow channel 37. The slot 31 is formed on the upper side of the water tank 30. The oscillation space 32 is defined in the water tank 30 for holding a liquid. The tank bottom 33 is located at the bottom of the oscillation space 32 and opens the perforation 34. One side of the air flow channel 37 protrudes into the oscillation space 32 of the water tank 30 to form an inner vent 371, and the other side of the air flow channel 37 extends out of the tank bottom 33 to form an outer vent 373. The electric fan 20 is mounted in the outer vent 373.

The water tank 30 is mounted inside the accommodation chamber 11. The outer vent 373 of the air flow channel 37 and the electric fan 20 are disposed in the accommodation chamber 11 in communication with the first air inlet 12. The first air inlet 12 provides a path for the electric fan 20 to inhale external air.

The tank cover 35 comprises a shield 355 and a first exhaust hole 357. The shape of the tank cover 35 matches the structure of the inner vent 371 in the water tank 30. The shield 355 forms a concave shape on the inner side of the peripheral edge of the tank cover 35, and the inner side of the concave shape of the shield 355 forms a sheltered space. The first exhaust hole 357 is formed on the other side of the tank cover 35. The tank cover 35 is detachably covered on the slot 31 and at the same time, the shield 355 of the tank cover 35 is covered over the inner vent 371. The inner vent 371 and the first exhaust hole 357 are commonly disposed in communication with the oscillation space 32.

The oscillation device 40 is mounted in the perforation 34, which is used to vibrate and atomize the liquid in the oscillation space 32.

The spray cover 60 comprises a spray hole 671. The spray cover 60 is detachably mounted on the opening 13 of the bottom housing 10 to shield the tank cover 35, so that the spray hole 671 communicates with the first exhaust hole 357 and the oscillation space 32 to form a spray outlet path.

The control unit 70 is mounted in the accommodation chamber 11 of the tank bottom 33, comprising circuit devices such as printed circuit board assembly (PCBA), and circuit means for driving the oscillation device 40. The control unit 70 is electrically connected with the oscillation device 40, the electric fan 20 or other lamps, electronics or appliances used in the in-car environment purification diffuser. It is used to control the switching or various action functions of the oscillation device 40 or the electric fan 20 or other lamps, electronics or appliances.

In this way, connect a power supply to the control unit 70, command to turn on the oscillation device 40 to oscillate the liquid in the oscillation space 32 (such as alkaline ionized water or various environmental cleaning and purification agents, etc., or add various natural sterilization and deodorant agents to the liquid for application) into atomized mist (fine water molecules). At the same time, the electric fan 20 continuously sends the air flow from the first air inlet 12 to the outer vent 373 to passthrough the inner vent 371 of the air flow channel 37. The airflow is blocked by the shield 355 from backflow, and it flows in the oscillation space 32 without directly flowing out of the first exhaust hole 357. It can effectively transport atomized alkaline ionized water (in the embodiment, the pH value of the hydroxide ionized water electrolyzed by pure water is about 10 to 12.5) and various mists (fine water molecules) from the oscillation space 32 through the first exhaust hole 357 and the spray cover 60 to the car internal space or the outside environment for use in sterilization, cleaning, deodorization, virus elimination, or air purification.

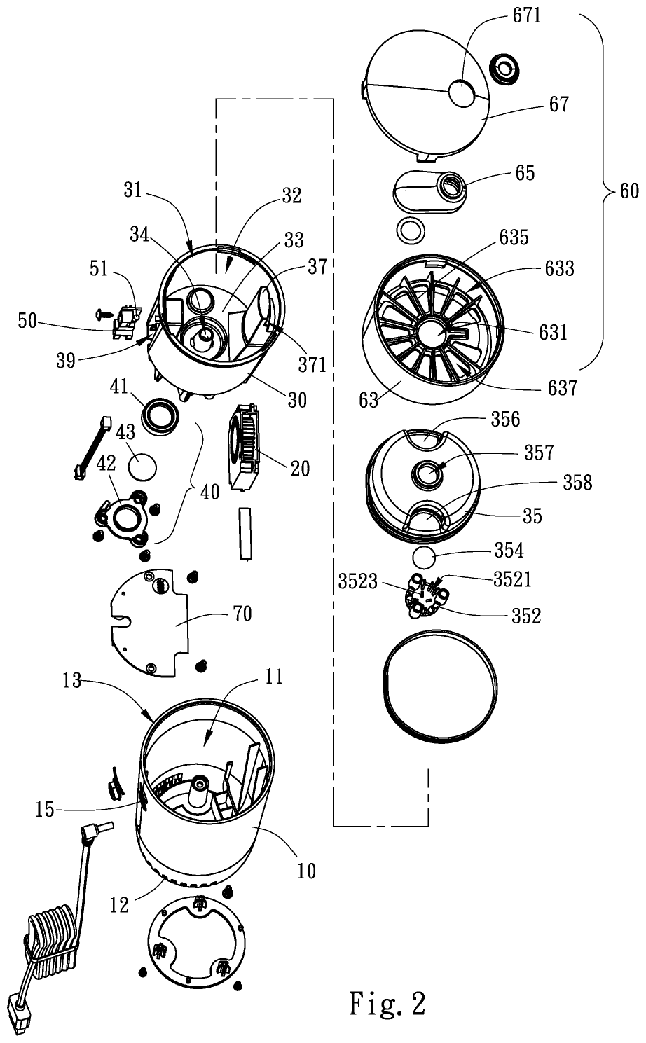

Referring to FIGS. 2-5, in some embodiments, the diameter of the outer vent 373 is larger than that of the inner vent 371, so that when the air flow produced by the electric fan 20 can flow from the outer vent 373 toward the inner vent 371 at a relatively higher speed and higher pressure into the oscillation space 32 to rapidly transport the mist oscillated by the oscillation device 45 to the spray hole 671 toward the space outside the diffuser. Therefore, the atomized water molecules that have increased in a large amount are less likely to stay in the atomization space and fall into the liquid.

Referring to FIGS. 2-5, in some embodiments, the oscillation device 40 comprises a waterproof ring 41, a fixing frame 42 and an oscillator 43. The oscillator 43 can be a ceramic piece, or metal piece such as iron piece or copper piece. The outer layer of the oscillator 43 has an acid-and-alkaline-resistant coating. The acid-and-alkaline-resistant coating is used to prevent, for example, alkaline ionized water or various environmental cleaning and purifying agents, or cleaning, sterilization, deodorization, and disinfectant liquids from corroding the oscillator 43 to cause damage.

In an embodiment, the acid-and-alkaline-resistant coating of the oscillator 43 has a film thickness of 0.5 to 1.5 microns. Preferably, the film thickness is about 1 micron. The acid-and-alkaline-resistant coating contains fluorine-containing polymer (fluoropolymer) or fluorine-containing oligomers or electronic metal coating agents commonly used in the electronics industry, etc. The oscillation device 40 is set in the perforation 34 at the bottom of the oscillation space 32. The oscillator 43 is mounted in the waterproof ring 41, and then the waterproof ring 41 is set in the perforation 34. Fix the oscillation device 40 by the fixing frame 42 located on the lower side of the waterproof ring 41 and coupled to the lower side of the perforation 34. The oscillator 43 of the present invention is protected by the acid-and-alkaline-resistant coating, and the alkaline ionized water electrolyzed by natural pure water (for example, the pH value of the hydroxide ionized water electrolyzed by pure water is about 10 to 12.5) can be used to clean, sterilize, deodorize and disinfect the environment in the car to purify the environmental space in the car. Wherein, the waterproof ring 41 can be, for example, a silicone ring, a rubber ring, or a plastic ring.

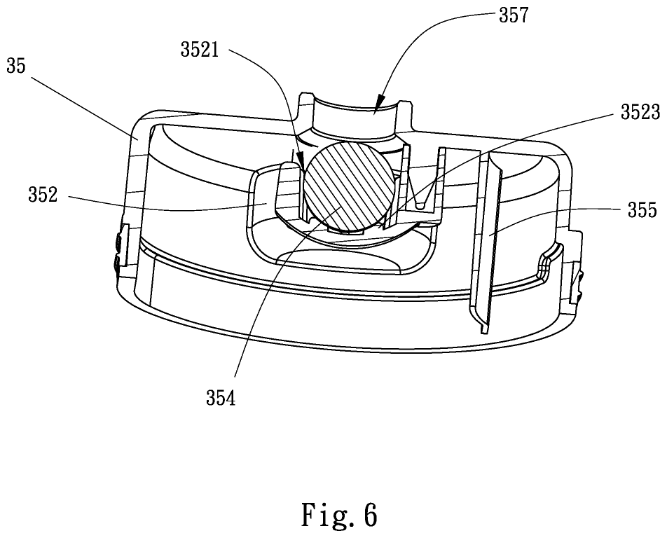

Referring to FIGS. 2-6, in some embodiments, the tank cover 35 covers the slot 31. The shield 355 covers the outer side of the inner vent 371 of the air flow channel 37. The inner vent 371 forms a height (distance) relative to the inner wall of the water tank 30. Thereby, the inner vent 371 with a special-shaped design of tubular protrusion in the water tank 30 has a height (distance) formed relative to the inner wall of the water tank 30. When the in-car environment purification diffuser is inclined, the liquid in the water tank 30 can avoid the part of the inner vent 371 and concentrate on the inner wall of the water tank 30 on both sides. Therefore, the inclined liquid is less likely to flow directly into the inner vent 371. It is difficult for liquid to enter the interior of the in-car environment purification diffuser, which helps to avoid damage to the electronic parts and electrical connection points of the control unit 70 and the electric fan 20. When replenishing liquids such as alkaline ionized water or various environmental cleaning and purifying agents in the oscillation space 32, the spray cover 60 can be easily opened from or covered on the opening 13 of the bottom housing 10, and the tank cover 35 can also be opened from or covered on the slot 31 of the water tank 30. The user can arbitrarily cover the spray cover 60 and tank cover 35 to the water tank 30 to replenish liquid. The structure is simple and convenient to use.

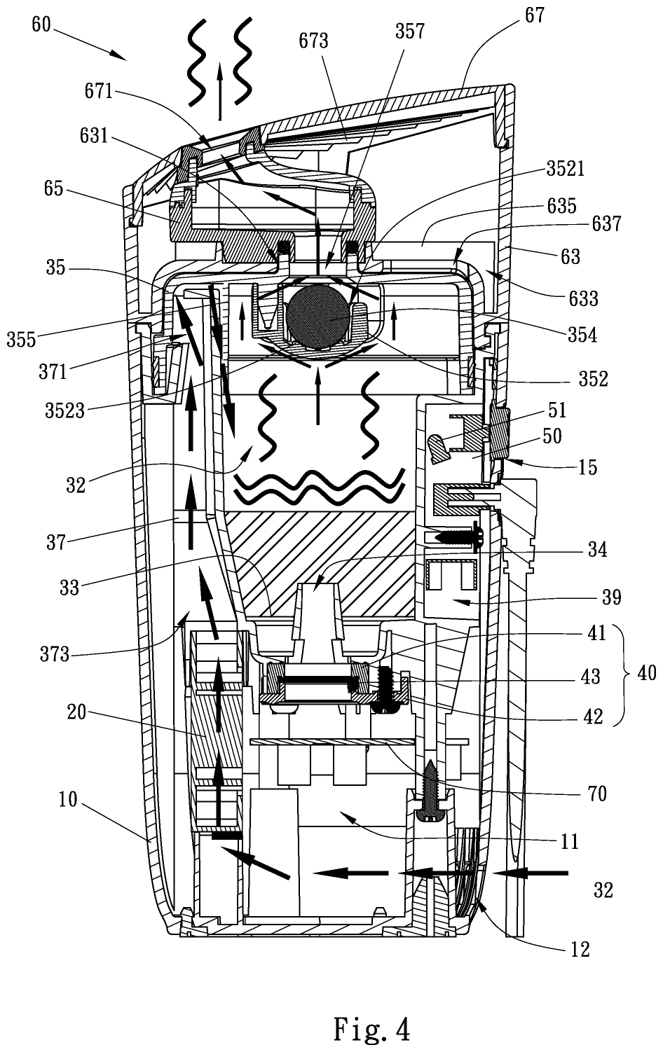

Referring to FIG. 2 and FIG. 4, the tank cover 35 also comprises a first arc portion 356 provided on one side of the circumference of the tank cover 35, and a second arc portion 358 provided on the other side of the circumference of the tank cover 35 opposite to the first arc portion 356. The first arc portion 356 and the second arc portion 358 are used to facilitate the user to manually mount the tank cover 35 to the slot 31 and detach it from the slot 31.

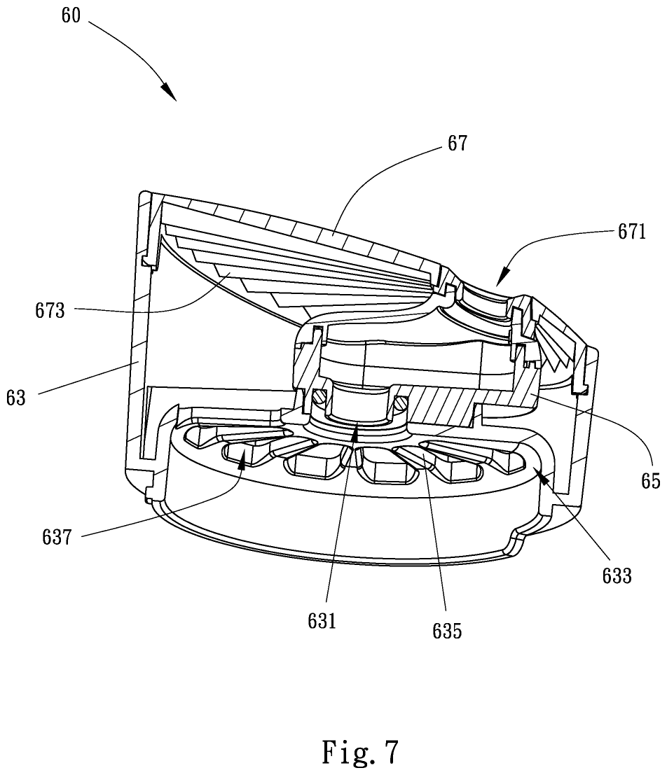

Referring to FIGS. 2-7, in one embodiment of the present invention, the spray cover 60 comprises a bottom cover 63, an air duct 65, and an umbrella-shaped panel 67. The bottom cover 63 is provided with a second exhaust hole 631. The spray hole 671 is formed in the umbrella-shaped panel 67. The air duct 65 has one side thereof connected to the upper side of the second exhaust hole 631, and an opposite side thereof connected to the lower side of the spray hole 671 of the umbrella-shaped panel 67. Thereby, the mist generated by alkaline ionized water in the oscillation space 32 and flowing from the first exhaust hole 357 through the second exhaust hole 631 of the spray cover 60 directly flows through the air duct 65 to the external space via the spray hole 671. Therefore, the mist will not flow into the inside of the diffuser to cause interference or damage.

In one embodiment of the present invention, as shown in FIGS. 1-5 and FIG. 7, the umbrella-shaped panel 67 is based on the Hakka culture paper umbrella skeleton structure and its visual appearance aesthetics. The umbrella-shaped panel 67 comprises a plurality of radial ribs 673, which radiate from the periphery of the spray hole 671 of the umbrella-shaped panel 67 to the edge of the umbrella-shaped panel 67. These radial ribs 673, like the frame of a paper umbrella, have support and decoration functions, which promote the umbrella-shaped panel 67 to have higher rigidity. At the same time, the production of the umbrella-shaped panel 67 can also reduce the material and weight efficiency. With these radial ribs 673, the umbrella-shaped panel 67 can be made thinner and lighter, and it is not easily damaged during assembly or in the process of dumping and impact in the car.

In some embodiments, these radial ribs 673 are arranged on the inner side of the umbrella-shaped panel 67 and radially extended from the from the periphery of the spray hole 671 of the umbrella-shaped panel 67 to the edge of the umbrella-shaped panel 67. The umbrella-shaped panel 67 can be selectively made of various translucent or transparent plastic materials, acrylic, glass, etc. This translucent or transparent umbrella-shaped panel 67 vaguely sees through the radial ribs 673, and the overall shape is like a paper umbrella with a decorative effect.

In one embodiment of the present invention, as shown in FIGS. 2, 3 and 7, the bottom cover 63 also comprises a grating 633, which comprises a plurality of bars 635 radially extended from around the second exhaust hole 631 to the inner wall of the bottom cover 63, and a plurality of light holes 637 formed between the bars 635. The bars 635 are, for example, fin-type bars, which are combined in the X-axis direction and occupy less space while maintaining a certain degree of rigidity. The bars 635 can also enable the light hole 637 to maintain a larger aperture. The grating 633 supports the periphery of the bottom cover 63 to increase the rigidity of the bottom cover 63, so that the bottom cover 63 is not easily deformed when it is operated or rotated.

Referring to FIGS. 1-7, in one embodiment of the present invention, the in-car environment purification diffuser further comprises a switch circuit 50. The switch circuit 50 comprises a power switch button and is electrically connected to the control unit 70. The water tank 30 also comprises a groove 39 that is recessed from the outside of the water tank 30 to the inside. A side hole 13 is opened on one side of the bottom housing 10. The switch circuit 50 is combined with the groove 39, and the power switch button of the switch circuit 50 is clamped in the side hole 13.

In some embodiments, a light source 51 is also incorporated in the groove 39. The light source 51 is a light-emitting device such as an LED lamp or a bulb, and the light source 51 is electrically connected to the control unit 70. The groove 39 and tank cover 35 are made of transparent or translucent plastic material, acrylic or glass, etc. The control unit 70 is electrically connected to an external power source to provide power supply to the light source 51. The light emitted by the light source 51 goes through the wall of the groove 39, the tank cover 35 and the light hole 637 to radiate on the transparent umbrella-shaped panel 67 to form a light decoration effect. The light passes through the transparent umbrella-shaped panel 67 is refracted by the radial ribs 673 to produce a skeleton charm of a paper umbrella and a light decoration effect. Combining the mist-spray effect and the lighting effect, the in-car environment purification diffuser can have a better atmosphere. In particular, the water tank 30 can also be additionally added with fragrance liquid to make the car interior sterilization, disinfection, and deodorization smell better.

In this way, the switch circuit 50 provides the button selection function and the switch signal is sent to the control unit 70. The control unit 70 is used to connect the power supply and provide button selection functions and switch signals to turn on or off the oscillating device 40, the electric fan 20 or the light source 51. The control unit 70 instructs to turn on the oscillation device 40 to generate atomized mist (fine water molecules) by oscillating the liquid in the oscillation space 32 of the tank cover 35. At the same time, the electric fan 20 continues to deliver the air flow from the first air inlet 12, through the air flow channel 39 into the shield 355 and then the oscillation space 32. The air flow transports the atomized mist (fine water molecules) through the first exhaust hole 357, the air duct 65 and the spray hole 671 to the external environment.

In some embodiments, the control unit 70 can be any of a variety of printed circuit board assemblies or electronic circuits, or various computer equipment such as computers or microprocessors, which can be used in the operation or control function of the in-car environment purification diffuser of the present invention.

Referring to FIGS. 2-6, in one embodiment of the present invention, the tank cover 35 further comprises a connection frame 352 and a ball 354. A depression 3521 and a plurality of bumps 3523 are formed in the middle of the connection frame 352, and the plurality of bumps 353 protrude from the bottom side of the depression 3521. The ball 354 can temporarily stay in the depression 3521 and abut on the bumps 5323. The depression 3521 and the ball 354 have an interference fit, and the bumps 5323 abut the bottom of the ball 354 so that the ball 354 will not be completely stuck in the depression 3521. The connection frame 352 is used to combine with the lower side of the first exhaust hole 357 of the tank cover 35. The ball 354 corresponds to the first exhaust hole 357, while the ball 354 and the first exhaust hole 357 maintain a gap. Thereby, the atomized water molecules generated in the oscillation space 32 flow through the connection frame 352 and the side or gap of the ball 354 and the first exhaust hole 357 to enter the spray cover 60. If the in-car environment purification diffuser is accidentally dumped during use, the ball 354 will move forward to block the first exhaust hole 357 to prevent the liquid in the water tank 30 from flowing out of the first exhaust hole 357 and spray hole 631 to the external environment, avoiding a large amount of liquid or aromatic liquid or chemical substances in the water tank 30 from directly flowing out of the in-car environment purification diffuser, which can maintain the clean and safe external environment of the in-car environment purification diffuser.

In one embodiment of the present invention, the in-car environment purification diffuser is used for sterilization or disinfection. The user will use cleaning, sterilizing, disinfecting or/and deodorizing liquids, for example, alkaline ionized water with a pH value of 10.0 or more, such as pH 10, 10.5, 11, 11.5, 12, 12.5 pure water electrolysis alkaline ionized water. In the preferred embodiment, the pH value of hydroxide ionized water, such as pure water electrolysis, is 10 to 12.5. Alkaline ionized water has a peeling effect, which can neutralize static electricity and float away from dirt. Alkalization can decompose oil or protein. Alkaline ionized water is more permeable than ordinary water and has a good cleaning and sterilization effect.

In the embodiment, the hydroxide ion water electrolyzed by pure water with a pH value of 10.5 to 12.5 is used. The hydroxide ion water is poured into the oscillation space 32 of the water tank 30. The user switches on the switch circuit 50 to provide power to the control unit 70, so that the control unit 70 drives the oscillation device 40 to vibrate, causing the alkaline ionized water in the oscillation space 32 to generate mist. At the same time, the electric fan 20 is turned on to drawing outside air from the first air inlet 12 of the bottom housing 10 through the air flow channel 37 to the shield 355 and the oscillation space 32, forcing the alkaline ionized water mist to flow from the oscillation space 32 through the first exhaust hole 357, the second exhaust hole 631, the air duct 65 and the spray hole 671 into the car inside space to sterilize, disinfect and deodorize the environment in the car. Eliminate odors in the car, purify the air in the car, etc. The in-car environment purification diffuser is safe to use and does not affect health.

Although the present invention has been described using the above-mentioned preferred embodiments, it is not intended to limit the scope of protection of the present invention. Any person skilled in the art can make various modifications and changes relative to the above-mentioned embodiments without departing from the spirit and scope of the present invention. Therefore, the scope of protection of the present invention is as defined by the claims.

What the invention claimed is:

1. An in-car environment purification diffuser, comprising a bottom housing, an electric fan, a water tank, an oscillation device, a spray cover, and a control unit, wherein:
said bottom housing comprises an accommodation chamber defined inside, a first air inlet provided on a bottom side of said accommodation chamber, and an opening formed on an upper edge of said accommodation chamber;
said water tank comprises a slot, an oscillation space, a tank bottom, a perforation, a tank cover and an air flow channel, said slot being formed on an upper side of said water tank, said oscillation space being defined in said water tank for holding a liquid, said tank bottom being located at a bottom of said oscillation space and opening said perforation, said air flow channel having one side thereof protruding into said oscillation space of said water tank to form an inner vent and an opposite side thereof extending out of said tank bottom to form an outer vent;
said electric fan is mounted in said outer vent;
said water tank is mounted inside said accommodation chamber;
said outer vent of said air flow channel and said electric fan are disposed in said accommodation chamber in communication with said first air inlet, which provides a path for said electric fan to inhale external air;
said tank cover comprises a shield and a first exhaust hole, said shield forming a concave shape on an inner side of the peripheral edge of said tank cover, said first exhaust hole being formed on an opposite side of said tank cover, said tank cover being detachably covered on said slot, said shield of said tank cover being covered over said inner vent, said inner vent and said first exhaust hole being commonly disposed in communication with said oscillation space;
said oscillation device is mounted in said perforation;
said spray cover comprises a spray hole, said spray cover being detachably mounted on said opening of said bottom housing to shield said tank cover, so that said spray hole communicates with said first exhaust hole and said oscillation space to form a spray outlet path;
said control unit is mounted in said accommodation chamber at one side of said tank bottom and electrically connected with said oscillation device and said electric fan.

2. The in-car environment purification diffuser as claimed in claim 1, wherein said oscillation device comprises a waterproof ring, a fixing frame and an oscillator, said oscillator being peripherally coated with an acid-and-alkaline-resistant coating.

3. The in-car environment purification diffuser as claimed in claim 2, wherein said acid-and-alkaline-resistant coating comprises a fluorine-containing polymer (fluoropolymer) or fluorine-containing oligomers or an electronic metal coating agent.

4. The in-car environment purification diffuser as claimed in claim 1, wherein said shield covers an outer side of said inner vent of said air flow channel, and said inner vent forms a height relative to an inner wall of said water tank.

5. The in-car environment purification diffuser as claimed in claim 4, wherein the diameter of said outer vent is larger than that of said inner vent.

6. The in-car environment purification diffuser as claimed in claim 1, wherein said spray cover comprises a bottom cover, an air duct and an umbrella-shaped panel, said bottom cover being provided with a second exhaust hole, said umbrella-shaped panel having said spray hole formed therein, said air duct having one side thereof connected to an upper side of said second exhaust hole and an opposite side thereof connected to a lower side of said spray hole of said umbrella-shaped panel.

7. The in-car environment purification diffuser as claimed in claim 6, wherein said umbrella-shaped panel comprises a plurality of radial ribs arranged on an inner side thereof and radially extended from said spray hole to the border edge of said umbrella-shaped panel, said umbrella-shaped panel being selectively made of a light-permeable transparent or translucent material.

8. The in-car environment purification diffuser as claimed in claim 7, wherein said bottom cover comprises a grating, said grating comprising a plurality of bars radially extended from said second exhaust hole to an inner wall of said bottom cover and a plurality of light holes formed between said bars.

9. The in-car environment purification diffuser as claimed in claim 8, wherein said water tank comprises a groove formed by recessing from an outer side of said water tank to an inner side of said water tank, and a light source mounted in said groove and electrically connected to said control unit, said groove and said tank cover being selectively made of a light-permeable transparent or translucent material.

10. The in-car environment purification diffuser as claimed in claim 3, wherein said spray cover comprises a bottom cover, an air duct and an umbrella-shaped panel, said bottom cover being provided with a second exhaust hole, said umbrella-shaped panel having said spray hole formed therein, said air duct having one side thereof connected to an upper side of said second exhaust hole and an opposite side thereof connected to a lower side of said spray hole of said umbrella-shaped panel.

11. The in-car environment purification diffuser as claimed in claim 10, wherein said umbrella-shaped panel comprises a plurality of radial ribs arranged on an inner side thereof and radially extended from said spray hole to the border edge of said umbrella-shaped panel, said umbrella-shaped panel being selectively made of a light-permeable transparent or translucent material.

12. The in-car environment purification diffuser as claimed in claim 11, wherein said bottom cover comprises a grating, said grating comprising a plurality of bars radially extended from said second exhaust hole to an inner wall of said bottom cover and a plurality of light holes formed between said bars.

13. The in-car environment purification diffuser as claimed in claim 12, wherein said water tank comprises a groove formed by recessing from an outer side of said water tank to an inner side of said water tank, and a light source mounted in said groove and electrically connected to said control unit, said groove and said tank cover being selectively made of a light-permeable transparent or translucent material.

14. The in-car environment purification diffuser as claimed in claim 1, wherein said tank cover further comprises a connection frame and a ball, said connection frame comprising a depression and a plurality of bumps formed in a middle thereof, said bumps protruding from a bottom side of said depression, said ball movably staying in said depression and abutting on said bumps, said depression and said ball have an interference fit, said connection frame being connected to a bottom side of said first exhaust hole of said tank cover, said ball corresponding to said first exhaust hole with a gap left between said ball and said first exhaust hole.

15. The in-car environment purification diffuser as claimed in claim 2, wherein said tank cover further comprises a connection frame and a ball, said connection frame comprising a depression and a plurality of bumps formed in a middle thereof, said bumps protruding from a bottom side of said depression, said ball movably staying in said depression and abutting on said bumps, said depression and said ball have an interference fit, said connection frame being connected to a bottom side of said first exhaust hole of said tank cover, said ball corresponding to said first exhaust hole with a gap left between said ball and said first exhaust hole.

16. The in-car environment purification diffuser as claimed in claim 3, wherein said tank cover further comprises a connection frame and a ball, said connection frame comprising a depression and a plurality of bumps formed in a middle thereof, said bumps protruding from a bottom side of said depression, said ball movably staying in said depression and abutting on said bumps, said depression and said ball have an interference fit, said connection frame being connected to a bottom side of said first exhaust hole of said tank cover, said ball corresponding to said first exhaust hole with a gap left between said ball and said first exhaust hole.
